(19) United States
(12) Patent Application Publication
(10) Pub. No.: US 2008/0269766 A1
(43) Pub. Date: Oct. 30, 2008

(54) INTRAVERTEBRAL REDUCTION DEVICE WITH RETENTION BALLS

(75) Inventor: Jeff R. Justis, Germantown, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC, INC., Warsaw, IN (US)

(21) Appl. No.: 11/741,855

(22) Filed: Apr. 30, 2007

(51) Int. Cl. A61B 17/58; A61B 17/00

(52) U.S. Cl. 606/99; 606/80; 606/92

(57) ABSTRACT

An intravertebral reduction device is disclosed and can include a handle assembly and a shaft assembly extending from the handle assembly. The shaft assembly can be configured to create a space within a bone and deliver one or more retention balls to the space within the bone.

### FIELD OF THE DISCLOSURE

The present disclosure relates generally to orthopedics and orthopedic surgery. More specifically, the present disclosure relates to intravertebral reduction devices.

### BACKGROUND

In human anatomy, the spine is a generally flexible column that can take tensile and compressive loads. The spine also allows bending motion and provides a place of attachment for keels, muscles and ligaments. Generally, the spine is divided into three sections: the cervical spine, the thoracic spine and the lumbar spine. The sections of the spine are made up of individual bones (vertebrae) that are separated from each other by intervertebral discs.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

Further, vertebra can be broken due to traumatic injury. In such a case, the broken bone may need to be reduced. Once the bone is reduced, it may be desirable to maintain the bone in the reduced position.

### BRIEF DESCRIPTION OF THE DRAWINGS

FIG. 1 is a lateral view of a portion of a vertebral column;

FIG. 2 is a lateral view of a pair of adjacent vertebrae;

FIG. 3 is a top plan view of a vertebra;

FIG. 4 is a perspective view of an intravertebral reduction device;

FIG. 5 is a detailed perspective view of the intravertebral reduction device in a retracted configuration;

FIG. 6 is a detailed perspective view of the intravertebral reduction device in an expanded configuration;

FIG. 7 is a plan view of the intravertebral reduction device partially disposed within a vertebra; and

FIG. 8 and FIG. 9 is a flow chart illustrating one method of using an intravertebral reduction device.

### DETAILED DESCRIPTION OF THE DRAWINGS

An intravertebral reduction device is disclosed and can include a handle assembly and a shaft assembly extending from the handle assembly. The shaft assembly can be configured to create a space within a bone and deliver one or more retention balls to the space within the bone.

In another embodiment, a method of treating bone is disclosed and can include drilling a hole within a bone and moving an intravertebral reduction device through the hole into the bone. Further, the method can include moving the intravertebral reduction device to an expanded configuration and moving the intravertebral reduction device to a retracted configuration. The method can also include loading a plurality of retention balls into the intravertebral reduction device.

In yet another embodiment, an intravertebral reduction device is disclosed and can include a hollow inner shaft formed with a retention ball release hole. The intravertebral reduction device can also include a hollow outer shaft disposed around the inner shaft. The outer shaft can include a first flexible portion and a second flexible portion. Also, the intravertebral reduction device can be moved between a retracted configuration and an expanded configuration.

### Description of Relevant Anatomy

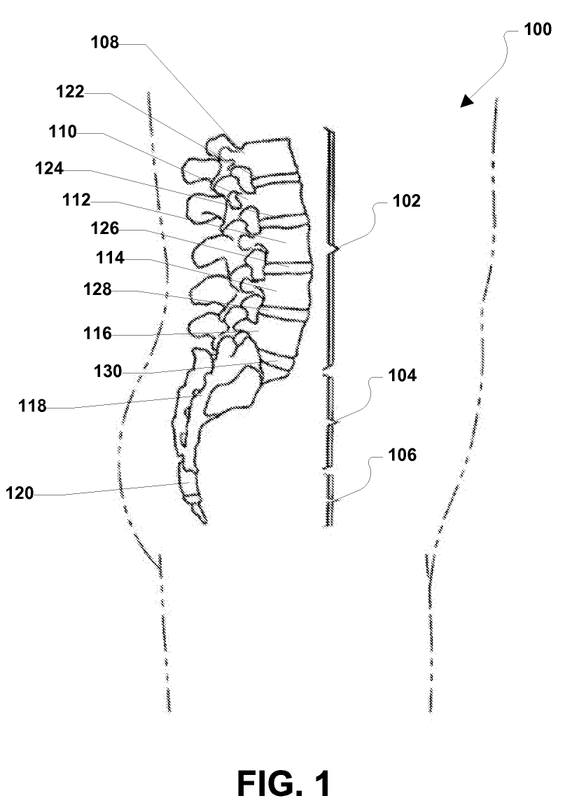

Referring initially to FIG. 1, a portion of a vertebral column, designated 100, is shown. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intravertebral lumbar disc 122 is disposed between the first lumbar vertebra 108 and the second lumbar vertebra 110. A second intravertebral lumbar disc 124 is disposed between the second lumbar vertebra 110 and the third lumbar vertebra 112. A third intravertebral lumbar disc 126 is disposed between the third lumbar vertebra 112 and the fourth lumbar vertebra 114. Further, a fourth intravertebral lumbar disc 128 is disposed between the fourth lumbar vertebra 114 and the fifth lumbar vertebra 116. Additionally, a fifth intravertebral lumbar disc 130 is disposed between the fifth lumbar vertebra 116 and the sacrum 118.

In a particular embodiment, if one of the intravertebral lumbar discs 122, 124, 126, 128, 130 is diseased, degenerated, damaged, or otherwise in need of repair, augmentation or treatment, that intravertebral lumbar disc 122, 124, 126, 128, 130 can be treated in accordance with one or more of the embodiments described herein.

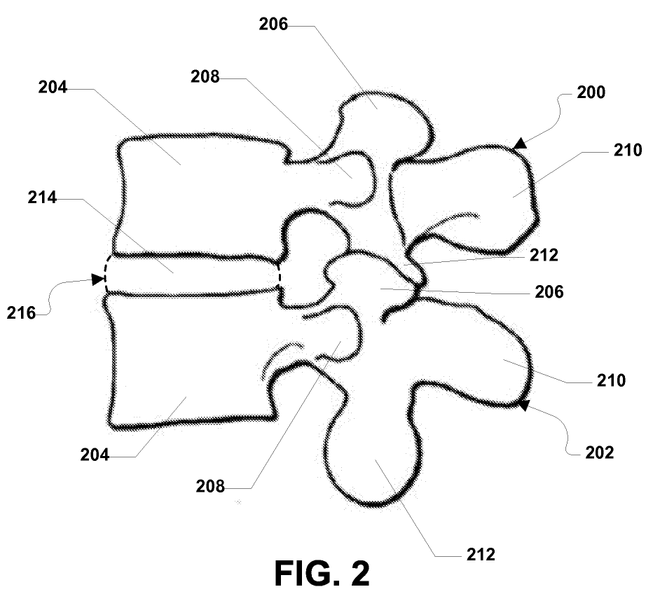

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral disc 216 between the superior vertebra 200 and the inferior vertebra 202.

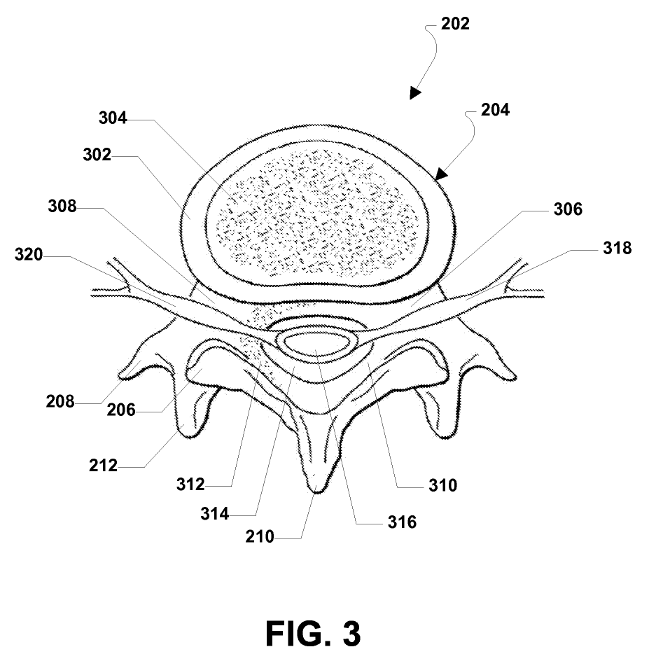

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

### Description of an Intravertebral Reduction Device

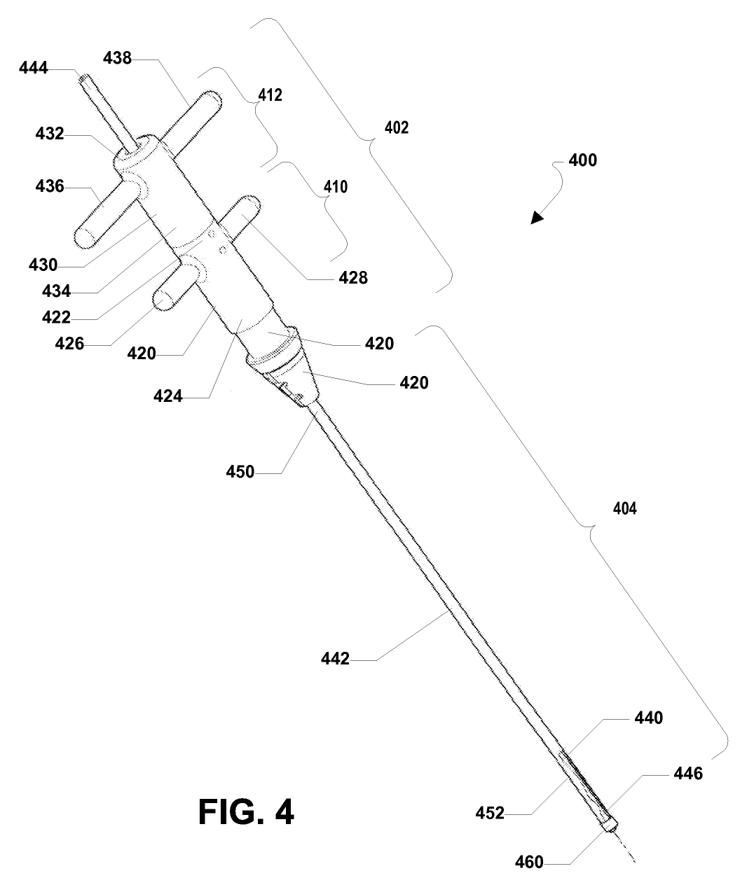

Referring to FIG. 4, an intravertebral reduction device is shown and is generally designated 400. As shown, the intravertebral reduction device 400 can include a handle assembly 402 and a shaft assembly 404 engaged with the handle assembly 402.

The handle assembly 402 can include a hilt 406 formed with a collar 408 that is configured to receive and engage the shaft assembly 404. Further, the handle assembly 402 can include a stationary handle 410 and a rotary handle 412 adjacent to the stationary handle 410. The stationary handle 410 can include a hollow, generally cylindrical body 420 having a proximal end 422 and a distal end 424. In a particular embodiment, the stationary handle 410 can be slidably disposed on the hilt 406. Also, a first arm 426 can extend from the body 420 of the stationary handle 410 near the proximal end 422 of the body 420. Also, a second arm 428 can extend from the body 420 of the stationary handle 410 near the proximal end 422 of the body 420. The second arm 428 can extend from the body 420 substantially opposite from the first arm 426.

As indicated in FIG. 4, the rotary handle 412 can include a generally cylindrical body 430 having a proximal end 432 and a distal end 434. As shown, a first arm 436 can extend from the body 430 of the rotary handle 412 near the proximal end 432 of the body 430. Also, a second arm 438 can extend from the body 430 of the rotary handle 412 near the proximal end 432 of the body 430. The second arm 438 can extend from the body 430 substantially opposite from the first arm 436.

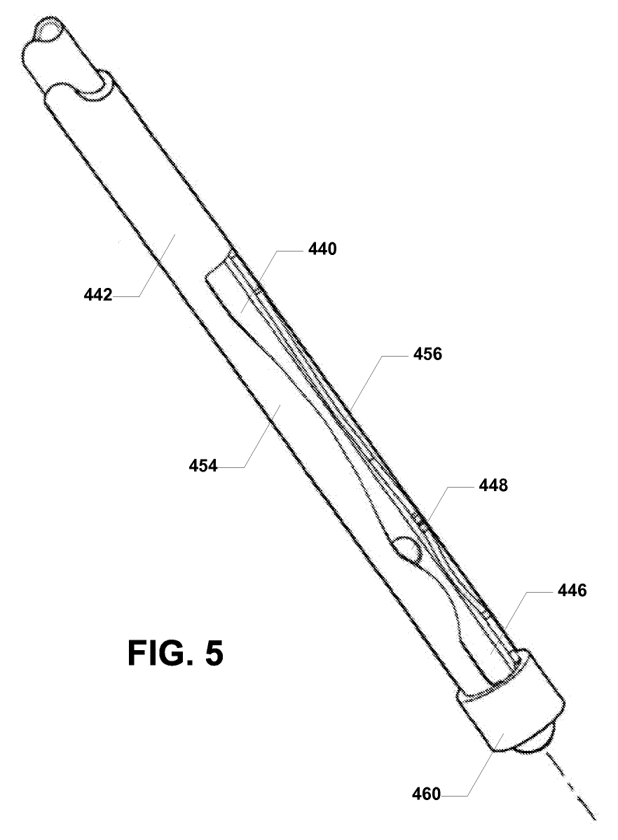

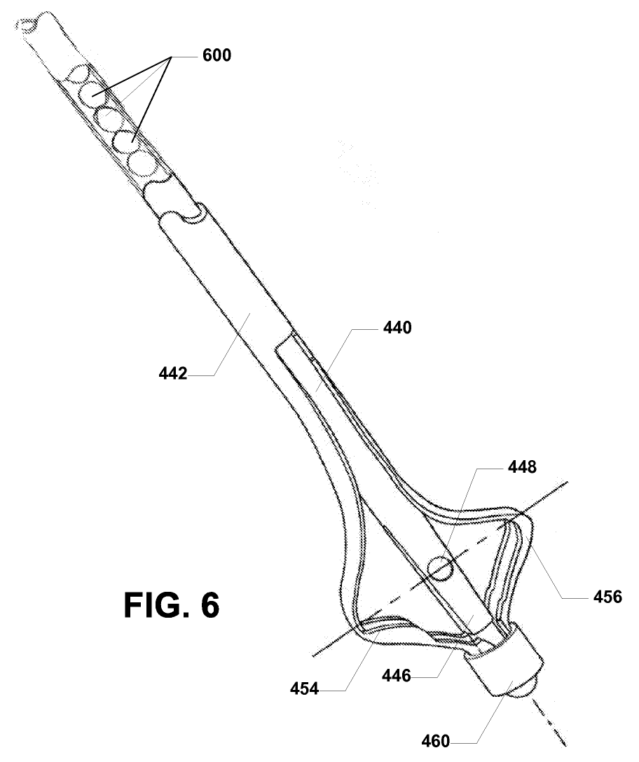

FIG. 4 shows that the shaft assembly 404 can include an inner shaft 440 and an outer shaft 442. The inner shaft 440 can be generally cylindrical and hollow. Further, the inner shaft 440 can include a proximal end 444 and a distal end 446. The inner shaft 440 can extend through the handle assembly 402 such that the proximal end 444 of the inner shaft 440 is distanced from the handle assembly 402. As shown in FIG. 5 and FIG. 6, the inner shaft 440 can be formed with a retention ball release hole 448 near the distal end 446 of the inner shaft 440.

The outer shaft 442 can also be generally cylindrical and hollow. Further, the outer shaft 442 can include a proximal end 450 and a distal end 452. FIG. 5 and FIG. 6 also show that the outer shaft 442 can include a first flexible portion 454 and a second flexible portion 456 near the distal end 452 of the outer shaft 442. The first flexible portion 454 can be substantially opposite to the second flexible portion 456. FIG. 4 through FIG. 6 also illustrate a compression ring 460 that can secure the distal end 452 of the outer shaft 442 to the distal end 446 of the inner shaft 440.

In a particular embodiment, the proximal end 450 of the outer shaft 442 can be coupled to the hilt 406 of the handle assembly 400. Further, the inner shaft 440 can extend through, and be threadably coupled to, the rotary handle 412. As the rotary handle 412 is rotated with respect to the stationary handle 410, e.g., clockwise, the inner shaft 440 can slide within the outer shaft 442 and cause the flexible portions 454, 456 of the outer shaft 442 to bend outward, as shown in FIG. 6. Accordingly, the intravertebral reduction device 400 can be moved between a retracted configuration, shown in FIG. 5, and an expanded configuration, shown in FIG. 6. In the retracted configuration, the flexible portions 454, 456 of the outer shaft 442 are substantially parallel to a longitudinal axis of the intravertebral reduction device 400. In the expanded configuration, the flexible portions 454, 456 of the outer shaft 442 bow outward relative to the longitudinal axis.

FIG. 6 further shows a plurality of retention balls 600 disposed within the inner shaft 440. The retention balls 600 can be moved through the inner shaft 400 and release from the inner shaft 440 via the retention ball release hole 448 formed in the inner shaft 440.

In a particular embodiment, the retention balls 600 can be made from one or more polymer materials. The polymer materials can include polyurethane materials, polyolefin materials, polyaryletherketone (PAEK) materials, or a combination thereof. Further, the polyolefin materials can include polypropylene, polyethylene, halogenated polyolefin, flouropolyolefin, or a combination thereof. The (PAEK) materials can include polyetherketone (PEK), polyetheretherketone (PEEK), polyetherketoneketone (PEKK), polyetherketoneetherketoneketone (PEKEKK), or a combination thereof.

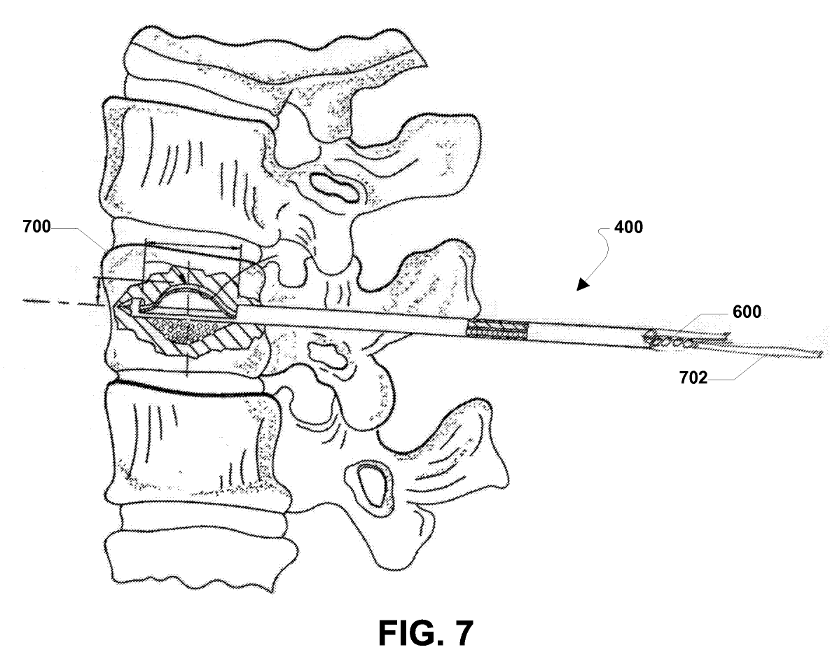

FIG. 7 depicts the intravertebral reduction device 400 engaged with a portion of a spinal column. As shown in FIG. 7, the end of the intravertebral reduction device 400 can be inserted into a bone, e.g., into a vertebral body 700. Thereafter, the intravertebral reduction device 400 can be expanded, as described herein, and rotated in order to reduce cancellous bone of the vertebral body 700 that has been damaged, or otherwise injured. After the cancellous bone of the vertebral body 700 is reduced, the intravertebral reduction device 400 can be retracted. Further, a plurality of retention balls 600 can be inserted into the space created within the vertebral body 700 in order to retain the cancellous bone in the reduced position. In a particular embodiment, a ramrod 702 can be used to move the retention balls 600 through the intravertebral reduction device 400, e.g., through the inner shaft 440. After a desired amount of retention balls 600 are installed within the vertebral body 700, a fusing material can be deposited around the retention balls 600 and cured.

### Description of a Method of Using an Intravertebral Reduction Device

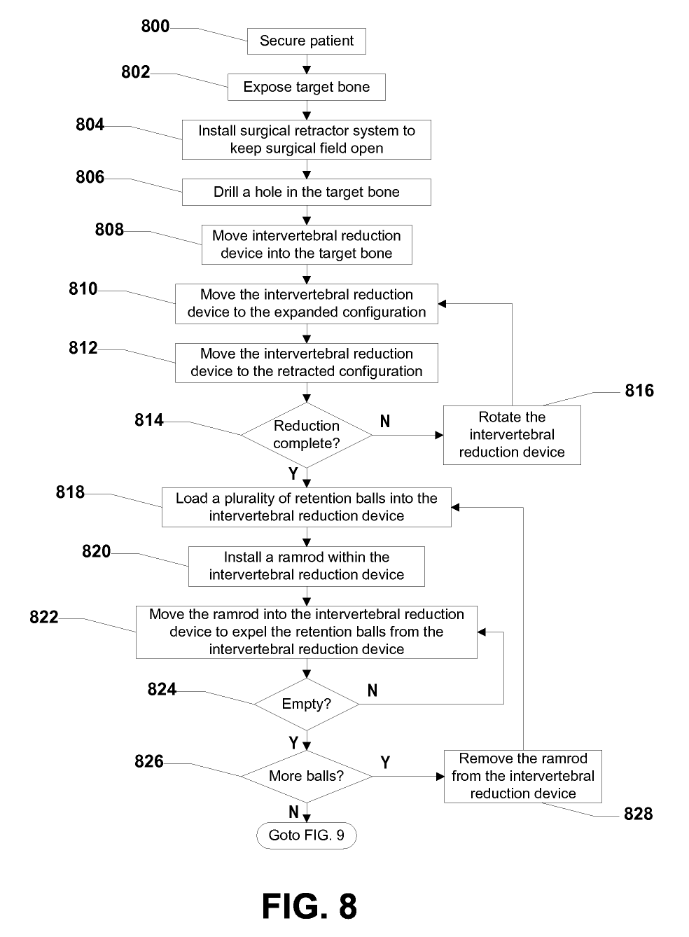

Referring to FIG. 8, a method of using an intravertebral reduction device is shown and commences at block 800. In a particular embodiment, the intravertebral reduction device can be the intravertebral reduction device described herein. At block 800, a patient can be secured on an operating table. For example, the patient can be secured in a prone position to allow a posterior approach to be used to access the patient's spinal column. Alternatively, the patient can be secured in a supine position to allow an anterior approach to be used to access the patient's spinal column. Further, the patient can be secured in a lateral decubitus position to allow a lateral approach to be used to access the patient's spinal column.

Moving to block 802, the target bone is exposed. Further, at block 804, a surgical retractor system can be installed to keep the surgical field open. For example, the surgical retractor system can be a surgical retractor system configured for posterior access to a spinal column. Alternatively, the surgical retractor system can be a surgical retractor system configured for anterior access to a spinal column. Also, the surgical retractor system can be a surgical retractor system configured for lateral access to a spinal column.

Proceeding to block 806, a hole can be drilled in the target bone. At block 808, the intravertebral reduction device can be moved into the target bone. For example, the distal end of the intravertebral reduction device can be moved into the target bone. Further, the target bone can be a vertebral body, an intravertebral space, or other tissue. Moving to block 810, the intravertebral reduction device can be moved to the expanded configuration. The intravertebral reduction device can be moved to the expanded configuration by rotating the rotatable handle relative to the stationary handle. Moving the intravertebral reduction device to the expanded configuration can compact or compress cancellous bone against the inner cortical wall of the vertebral body. Further, a cavity can be formed within the cancellous bone. Compacting the cancellous bone can cause an outward force to be exerted ion the inner surface of the cortical wall. The outward force can elevate or push broken, or otherwise compressed bone, back to, or substantially near, a pre-fracture position or another desired position.

After the intravertebral reduction device is moved to the expanded configuration and the fracture is reduced, the method can proceed to block 812 and the intravertebral reduction device can be moved to the retracted configuration. Thereafter, at decision step 814, it can be determined whether the reduction is complete. If the reduction is not complete, the method can proceed to block 816 and the intravertebral reduction device can be rotated within the target bone. The method can then return to block 810 and continue as described herein.

If the reduction is complete, the method can proceed to block 818 and a plurality of retention balls can be loaded into the intravertebral reduction device. At block 820, a ramrod can be installed within the intravertebral reduction device. Next, at block 822, the ramrod can be moved into the intravertebral reduction device to expel the retention balls from the intravertebral reduction device.

Continuing to decision step 824, it is determined whether the intravertebral reduction device is empty. If not, the method can return to block 822 and continue as described. If the intravertebral reduction device is empty, the method can proceed to decision step 826. At decision step 826, it can be determined whether more retention balls are necessary. If more retention balls are necessary, the method can move to block 828 and the ramrod can be removed within the intravertebral reduction device. The method can then return to block 818 and continue as described herein.

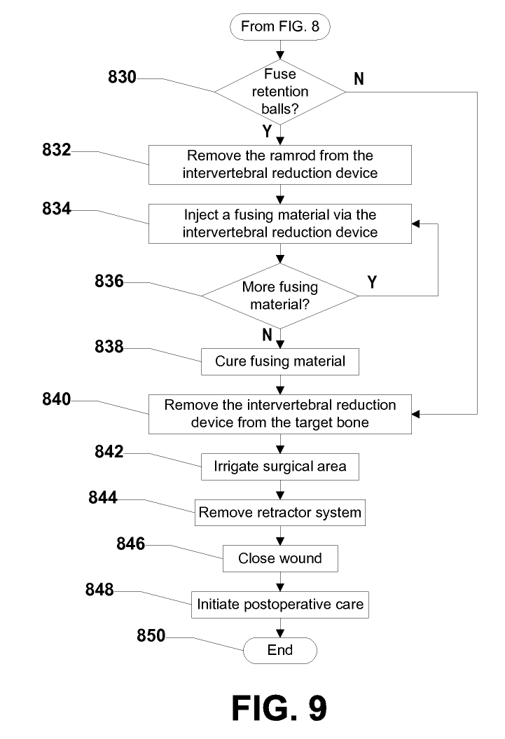

Returning to decision step 826, if more retention balls are not necessary, the method can proceed to decision step 830, shown in FIG. 9. At decision step 830, it can be determined whether to fuse the retention balls. If it is determined to fuse the retention balls, the method can proceed to block 832 and the ramrod can be removed from the intravertebral reduction device. At block 834, a fusing material can be injected into the retention balls within the target bone via the intravertebral reduction device, e.g., through the inner tube through which the retention balls were installed. The fusing material can be bone cement, a polymer, another material, or a combination thereof.

At decision step 836, it can be determined whether more fusing material is desirable. If so, the method can return to block 834 and continue as described herein. If more fusing material is not desired, the method can continue to block 838 and the fusing material can be cured. The fusing material can be cured by applying energy to the fusing material or by simply allowing the fusing material to cure naturally. Further, the curing energy can be applied to the fusing material via the intravertebral reduction device.

After the fusing material is cured, the method can move to block 840 and the intravertebral reduction device can be removed from the target bone. At block 842, the surgical space can be irrigated. Further, at block 844, the retractor system can be removed. At block 846, the surgical wound can be closed. The surgical wound can be closed using sutures, surgical staples, or any other surgical technique well known in the art. Moving to block 848, postoperative care can be initiated. The method can end at state 850.

### CONCLUSION

With the configuration of structure described above, the intravertebral reduction device provides a device that can be used to reduce damaged bone, e.g., a vertebra, a femur, a fibula, a tibia, etc. After the bone is reduced, a plurality of retention balls can be inserted into the reduced bone in order to maintain the bone in the reduced position. A fusing material can be injected into the retention balls in order to fuse the retention balls to each other.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. An intravertebral reduction device, comprising:
a handle assembly; and
a shaft assembly extending from the handle assembly, wherein the shaft assembly is configured to create a space within a bone and deliver one or more retention balls to the space within the bone.

2. The intravertebral reduction device of claim 1, wherein the shaft assembly includes an inner shaft and an outer shaft slidably disposed around the inner shaft.

3. The intravertebral reduction device of claim 2, wherein the outer shaft includes a first flexible portion and a second flexible portion.

4. The intravertebral reduction device of claim 3, wherein the intravertebral reduction device is movable between a retracted configuration and an expanded configuration.

5. The intravertebral reduction device of claim 4, wherein in the retracted configuration the first flexible portion and the second flexible portion are substantially parallel to a longitudinal axis of the intravertebral reduction device.

6. The intravertebral reduction device of claim 5, wherein in the expanded configuration the first flexible portion and the second flexible portion are bowed outward relative to the longitudinal axis of the intravertebral reduction device.

7. The intravertebral reduction device of claim 2, wherein the inner shaft is hollow and wherein the inner shaft is configured to allow the delivery of a plurality of retention balls to the space within the bone.

8. The intravertebral reduction device of claim 2, wherein the handle assembly includes a stationary handle attached to the outer shaft.

9. The intravertebral reduction device of claim 8, wherein the handle assembly includes a rotatable handle threadably engaged with the inner shaft.

10. The intravertebral reduction device of claim 9, wherein the rotatable handle is configured to rotate with respect to the stationary handle and slide the inner shaft relative to the outer shaft.

11. A method of treating bone, the method comprising:
drilling a hole within a bone;
moving an intravertebral reduction device through the hole into the bone;
moving the intravertebral reduction device to an expanded configuration;
moving the intravertebral reduction device to a retracted configuration; and
loading a plurality of retention balls into the intravertebral reduction device.

12. The method of claim 11, further comprising:
installing a ramrod within the intravertebral reduction device.

13. The method of claim 12, further comprising:
moving the ramrod into the intravertebral reduction device to expel the retention balls from the intravertebral reduction device and into the bone.

14. The method of claim 13, further comprising:
removing the ramrod from the intravertebral reduction device.

15. The method of claim 14, further comprising:
injecting a fusing material into the bone via the intravertebral reduction device.

16. The method of claim 15, further comprising:
curing the fusing material.

17. An intravertebral reduction device, comprising:
a hollow inner shaft formed with a retention ball release hole; and
a hollow outer shaft disposed around the inner shaft, wherein the outer shaft includes a first flexible portion and a second flexible portion and wherein the intravertebral reduction device is movable between a retracted configuration and an expanded configuration.

18. The intravertebral reduction device of claim 17, wherein in the retracted configuration the first flexible portion and the second flexible portion are substantially parallel to a longitudinal axis of the intravertebral reduction device.

19. The intravertebral reduction device of claim 18, wherein in the retracted configuration a plurality of retention balls can be moved through the inner shaft and expelled from the retention ball release hole.

20. The intravertebral reduction device of claim 19, wherein in the expanded configuration the first flexible portion and the second flexible portion are bowed outward relative to the longitudinal axis of the intravertebral reduction device.

21. The intravertebral reduction device of claim 20, further comprising a stationary handle attached to the outer shaft.

22. The intravertebral reduction device of claim 21, further comprising a rotatable handle threadably engaged with the inner shaft.

23. The intravertebral reduction device of claim 22, wherein the rotatable handle is configured to rotate with respect to the stationary handle and slide the inner shaft relative to the outer shaft.

24. The intravertebral reduction device of claim 23, wherein as the inner shaft slides relative to the outer shaft, the intravertebral reduction device moves to the expanded configuration.
